(19) United States
(12) Patent Application Publication
(10) Pub. No.: US 2006/0195089 A1
(43) Pub. Date: Aug. 31, 2006

(54) Spinal plating and intervertebral support systems and methods

(76) Inventors: Jean-Charles LeHuec, Pessac (FR); Curtis A. Dickman, Phoenix, AZ (US); Kent Anderson, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/050,589

(22) Filed: Feb. 3, 2005

(51) Int. Cl. A61B 17/58; A61F 2/30

(52) U.S. Cl. 606/61

(57) ABSTRACT

A plating system for stabilizing at least one level of the spinal column includes a plate member having a first portion with a bottom surface extending between opposite sides of the plate member. The bottom surface is positionable extradiscally along first and second vertebrae for engagement of the plate member thereto. A second portion of the plate member extends from one side of the first portion and away from the second side of the first portion. The second portion is received in the disc space between the first and second vertebrae and intradiscally engageable to at least one of the first and second vertebra.

### BACKGROUND

Various types of plating devices and systems have been used to stabilize portions of bones including the spine. Spinal stabilization techniques have employed plating on the posterior, anterior, lateral, postero-lateral or antero-lateral portions of a spinal column segment. Such plating systems can provide fixation of a spinal column segment for the repair of injured or diseased vertebrae, intervertebral discs, and other elements of the spinal column. Such plating systems can also be employed alone or in combination with other implants, such as interbody fusion devices.

While spinal plating systems and other bone fixation systems are known, the need remains for additional improvements. The present invention is directed to satisfying this need, among others.

### SUMMARY

According to one aspect, there is provided a spinal plating system comprising a plate member having a first portion positionable along first and second vertebrae and engageable to each of the first and second vertebrae with anchors located outside the disc space between the first and second vertebrae. The plate member includes a second portion extending from a side of the first portion that is received in the disc space between the first and second vertebrae when the first portion is engaged to the first and second vertebrae. The second portion is engageable with at least one of the first and second vertebra with a third anchor positioned in the disc space.

According to another aspect, a spinal plating system includes a plate member having a first portion and a second portion. Each of the portions include a top surface and a bottom surface extending between cephalad and caudal ends and first and second sides. The bottom surface of the first portion is positionable along first and second vertebrae when the plate member is engaged to the first and second vertebrae. The second portion extends medially from the first side of the first portion with the bottom surface of the second portion offset from the bottom surface portion of the first portion into the spinal disc space when the plate member is engaged to the first and second vertebrae.

According to a further aspect, a spinal plating system comprises a plate member having a first portion positionable extradiscally along first and second vertebrae and engageable thereto at a location offset laterally from the sagittal plane and a second portion extending medially from the first portion and positionable intradiscally between the first and second vertebrae and engageable thereto at a location adjacent the sagittal plane.

According to another aspect, there is provided a spinal plating system including a plate member having a Y-shape. A first portion of the plate member corresponds to an upper portion of the Y-shape and is positionable extradiscally between adjacent vertebra and engageable to the adjacent vertebrae with respective ones of first and second anchors. A second portion of the plate member corresponds to a lower leg of the Y-shape and is positionable intradiscally between the adjacent vertebrae and engageable to at least one of the adjacent vertebrae with at least one anchor.

According to another aspect, a method for stabilizing first and second vertebrae of a spinal column comprises: providing a plate member having a first portion and a second portion extending from a side of the first portion; positioning the first portion along the first and second vertebrae at a location offset laterally from the sagittal plane with the second portion in the disc space between the first and second vertebrae; engaging the first portion to each of the first and second vertebrae with respective ones of first and second anchors; and engaging the second portion to at least one of the first and second vertebrae with a third anchor extending through an endplate of the at least one vertebra.

According to yet another aspect, a method for stabilizing first and second vertebrae of the spinal column comprises: positioning a first portion of a plate member extradiscally along first and second vertebrae at a location offset laterally from the sagittal plane; positioning a second portion of the plate member intradiscally between the first and second vertebra at a location extending from a first side of the first portion toward the sagittal plane; engaging the first portion to the first and second vertebrae; and engaging the second portion to at least one of the first and second vertebrae.

These and other aspects will be discussed further below.

### BRIEF DESCRIPTION OF THE DRAWINGS

FIG. 1 is a perspective view showing engagement of the plating system to first and second vertebrae of a spinal column segment.

FIG. 2 is a perspective view looking toward the top of a plate member comprising the plating system of FIG. 1.

FIG. 3 is a perspective view looking toward the bottom of the plate member of FIG. 2.

FIG. 4 is a top view of the plating system of FIG. 1 including anchors through the plate holes.

FIG. 5 is a caudal end elevation view of the plating system of FIG. 4.

FIG. 6 is a left side elevation view of the plating system of FIG. 4.

FIG. 7 is a right side elevation view of the plating system of FIG. 4.

FIG. 8 is a caudal end elevation view of the plate member of FIG. 2.

FIG. 9 is a section view through line 9-9 of FIG. 8.

FIG. 10 is a right side elevation view of the plate member of FIG. 8.

FIG. 11 is a section view through line 11-11 of FIG. 10.

FIG. 12 is a top view of the plate member of FIG. 8 looking in the direction of arrow 50.

FIG. 13 is a top view of another embodiment plate member.

FIG. 14 is, when viewed in the same orientation as the FIG. 1 plate embodiment, a right side elevation view of the plate of FIG. 13.

FIG. 15 is a top view of another embodiment plating system without anchors.

FIG. 16 is a perspective view looking toward the top of the plating system of FIG. 15.

FIG. 17 is a top view of a plate member of the plating system of FIG. 15.

FIG. 18 is a caudal end elevation view of the plate member of FIG. 17.

FIG. 19 is a section view through line 19-19 of FIG. 17.

FIG. 20 is a section view through line 20-20 of FIG. 18.

FIG. 21 is an elevation view of one embodiment retaining member.

FIG. 22 is a section view through line 22-22 of FIG. 21.

FIG. 23 is an elevation view of another embodiment retaining member.

FIG. 24 is a section view through line 24-24 of FIG. 23.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and any such further applications of the principles of the invention as illustrated therein are contemplated as would normally occur to one skilled in the art to which the invention relates.

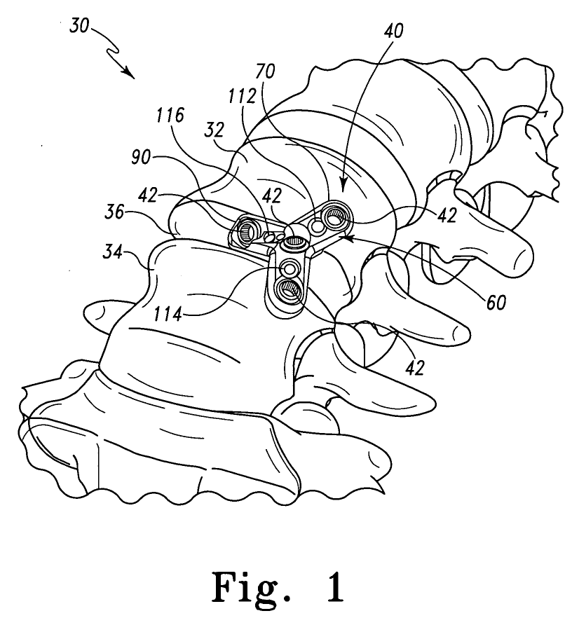

Referring to FIG. 1 there is shown a spinal column segment 30 having an upper or cephaladly positioned vertebra 32, a lower or caudally positioned vertebra 34, and a disc space 36 therebetween. In the illustrated embodiment, vertebrae 32, 34 correspond to the lumbar region of the spine, although vertebrae 32, 34 could be located along any region of the spinal column, including the cervical, thoracic, lumbar, and sacral regions. As used herein, upper refers to a cephalad direction or orientation, and lower refers to a caudal direction or orientation. Bottom refers to a direction or orientation toward the vertebrae, and top refers to a direction or orientation away from the vertebrae.

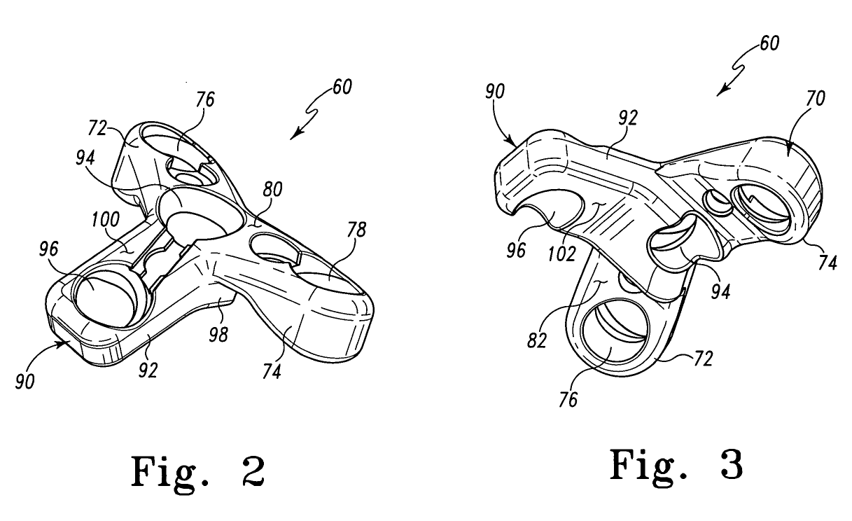

A plating system 40 includes a number of anchors 42 securing a plate member 60 to vertebrae 32, 34. As shown further in FIGS. 2-3, plate member 60 includes a first portion 70 extending extradiscally between vertebrae 32, 34 and a second portion 90 extending from one side of first portion 70 and positioned intradiscally between vertebrae 32, 34 in disc space 36. A first pair of the anchors 42 secure first portion 70 of plate member 60 to respective ones of the vertebrae 32, 34 from a location outside disc space 36. A second pair of the anchors 42 secure second portion 90 of plate member 60 to respective ones of the vertebrae 32, 34 from a location within disc space 36.

The location and positioning of plate member 60 and anchors 42 relative to spinal column segment 30 allow first portion 70 to be engaged extradiscally and antero-laterally along spinal column segment 30 to avoid the great vessels and other anatomical structures along the anterior portion of the spinal column. Furthermore, second portion 90 extends into the disc space and is offset from the top surface of first portion 70 to allow engagement of second portion 90 on or near the spinal midline. The combined extradiscal and intradiscal configuration of plate member 60 minimizes or eliminates protrusion of second portion 90 of plating system 40 from the disc space 36. Rigid stabilization of vertebrae 32, 34 can be achieved with plating system 40 while minimizing the invasiveness into the anterior or other anatomical structures while maintaining the ability to provide vertebral engagement with anchors adjacent the spinal midline and also with anchors engaged extradiscally and antero-laterally or laterally to the vertebrae 32, 34.

Plate member 60 includes first portion 70 having an upper end portion 72 positionable at least in part along upper vertebrae 32, and a lower end portion 74 positionable at least in part along lower vertebra 34. Upper end portion 72 includes an upper hole 76 extending between top and bottom surfaces 80, 82 of first portion 70. Upper hole 76 receives one of the anchors 42 to engage first portion 70 to upper vertebra 32. Lower end portion 74 includes a lower hole 78 extending between and opening at top and bottom surfaces 80, 82 of first portion 70. Lower hole 78 receives another of the anchors 42 to secure plate member 60 to lower vertebra 34.

Plate member 60 further includes second portion 90 having connecting portion 98 connected to or formed with first portion 70 along an intermediate side thereof. Connecting portion 98 is located between upper and lower end portions 72, 74 at the junction of first and second portions 70, 90. Intradiscal portion 92 extends in a direction away from first portion 70 for access thereto for placement of anchors to engage second portion 90 to at least one of the adjacent vertebrae. In the illustrated embodiment, intradiscal portion 92 includes an intradiscal hole 96 extending between and opening at top and bottom surfaces 100, 102 to receive an anchor 42 to intradiscally engage second portion 90 to one of the adjacent vertebrae 32, 34. A central hole 94 is provided at connecting portion 98, and receives an anchor 42 that extends intradiscally to secure plate member 60 to the other of the adjacent vertebrae 32, 34.

It is contemplated that intradiscal portion 92 and intradiscal hole 96 can be configured and positioned so as to be wholly contained within the disc space 36. It is also contemplated that intradiscal portion 92 and hole 96 can be configured and positioned to partially extend into the disc space 36, with top surface 100 being aligned at the outer margins of the vertebral endplates or protruding anteriorly therefrom. In one embodiment, at least a portion of either second portion 90 or the anchors 42 engaging second portion 90 to the adjacent vertebrae are received in disc space 36.

Other embodiments contemplate plate members adapted to extend extradiscally along three or more vertebrae. Such plate members can include one or more additional second portions for positioning intradiscally between vertebrae of the one or more next adjacent vertebral levels. Still other embodiments contemplate engagement of multiple plating systems along multiple vertebral levels, or the placement of multiple plating systems on a single vertebral level. Still other embodiments contemplate two or more plates along one or more vertebral levels that are linked to one another by an interface between the plates, by a linking member, by a fastener, or by any combination thereof. Any one or more of the vertebral levels may include interbody fusion devices, implants, artificial discs, bone graft, or other devices or materials as may be desired.

It is contemplated that plating system 40 can be provided with retaining systems to resist anchors 42 from backing out from plate member 60 when engaged to vertebrae 32, 34. For example, retaining fasteners 112, 114 can be engaged to plate member 60 along or adjacent top surface 80 and adjacent respective ones of holes 76, 78 in overlapping arrangement therewith to prevent anchors 42 from backing out thereof. A retaining plate 116 can be engaged to plate member 60 along top surface 100. Retaining plate 116 extends between and overlaps holes 94, 96 to prevent anchors 42 from backing out therefrom. Retaining plate 116 and retaining fasteners 112, 114 can be recessed at least partially below the respective adjacent top surface to minimize protrusion of the retaining systems into the adjacent anatomy.

Other embodiments contemplate other anchor retaining mechanisms to prevent or resist anchor backout, including sliding plates, rotating plates, locking screws with retaining washers, snap rings, resiliently deformable members integrally formed with or separately attached to the plate member, shape memory devices, cover plates that completely or substantially cover all or a portion of plate member 60, for example. Such retaining mechanisms can either be associated with a single hole or can be associated with multiple holes.

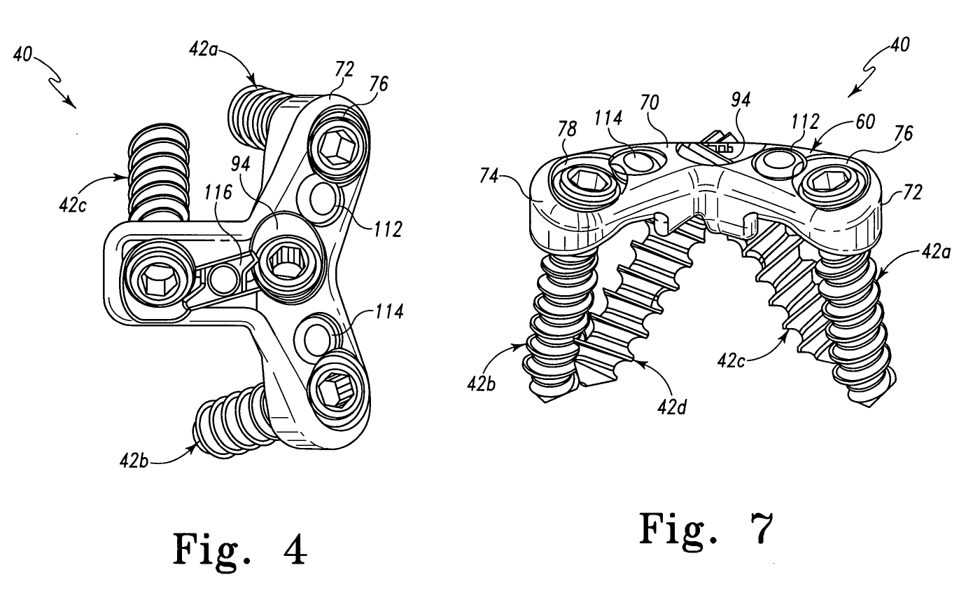

Referring now to FIGS. 4-7, there are shown various views of plating system 40 with anchors 42 positioned through the holes thereof in one embodiment engagement orientation with the respective vertebrae 32, 34. Upper hole 76 receives an upper anchor 42a and is configured so that upper anchor 42a extends cephaladly and medially when positioned therethrough and plate member 60 is positioned on vertebrae 32, 34 as shown in FIG. 1. Lower hole 78 receives a lower anchor 42b that extends from plate member 60 caudally and medially when positioned therethrough and plate member 60 is positioned on vertebrae 32, 34 as shown in FIG. 1. The cephalad and caudal orientations of holes 76, 78 allows the footprint of first portion 70 of plate member 60 to be minimized in the cephalad and caudal directions while obtaining bony purchase along a major portion of the depth of the vertebral body with bone engaging threads along anchors 42a, 42b.

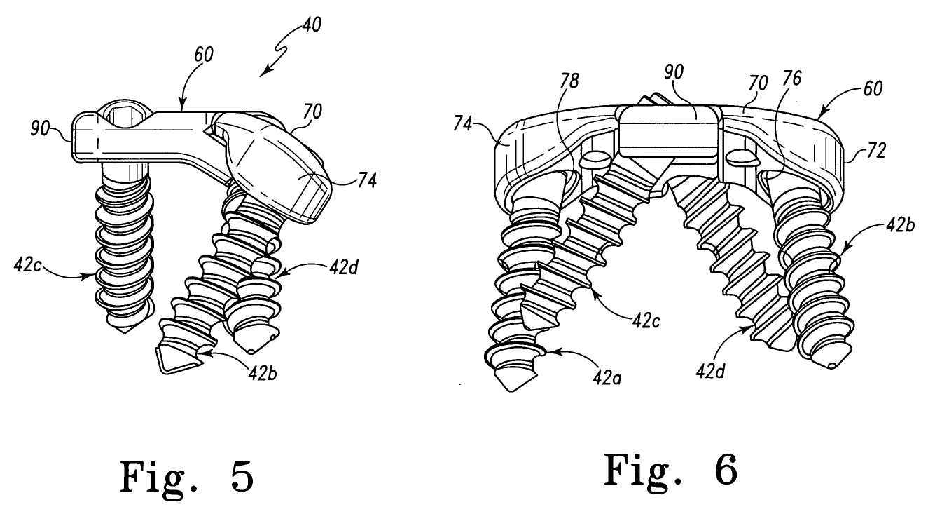

Intradiscal hole 96 includes anchor 42c that extends cephaladly for engagement with the upper vertebra 32 through its endplate. Central hole 94 includes anchor 42d that extends caudally for engagement with the lower vertebra 34 through its endplate. Anchors 42c and 42d extend generally parallel to the sagittal plane of the spinal column when plate member 60 is positioned on vertebrae 32, 34 in the orientation shown in FIG. 1, for example, where first portion 70 is positioned laterally or antero-laterally and second portion 90 extends intradiscally toward the sagittal plane so that intradiscal hole 96 is aligned with or adjacent the sagittal plane. Anchors 42a and 42b are angled medially such that their leading ends are positioned more medially than the leading end of anchor 42d, as shown in FIG. 5.

Each of the holes 76, 78, 94, 96 can be provided with spherically shaped recesses thereabout to receive the underside of the head of the anchor to allow the head of the anchor to be recessed relative to the adjacent top surface 80, 100 of plate member 60. It is still further contemplated that engagement of the anchors 42 with the respective holes in plate member 60 can provide a fixed angle orientation for the anchors, or allow at least some multi-axial pivoting or placement of the respective anchor 42 relative to plate member 60.

It is also contemplated that plate member 60 could be positioned at other locations relative to vertebrae 32, 34, including positioning first portion 70 anteriorly with second portion 90 extending to an antero-lateral or lateral disc space location. In another embodiment, first portion 70 is positioned laterally or antero-laterally and second portion 90 extends to an antero-lateral or lateral location in the disc space on the opposite side of the sagittal plane. It is further contemplated that the direction of angulation of holes 94, 96 could be reversed so that anchor 42c engages lower vertebra 34 and anchor 42d engages upper vertebra 32. Other angular orientations and angular relationships between the centerlines of the plate holes and the anchors positioned therein are also contemplated. It is further contemplated that either or both of first and second portions 70, 90 can be provided with a single hole, a pair of holes, or with three or more holes.

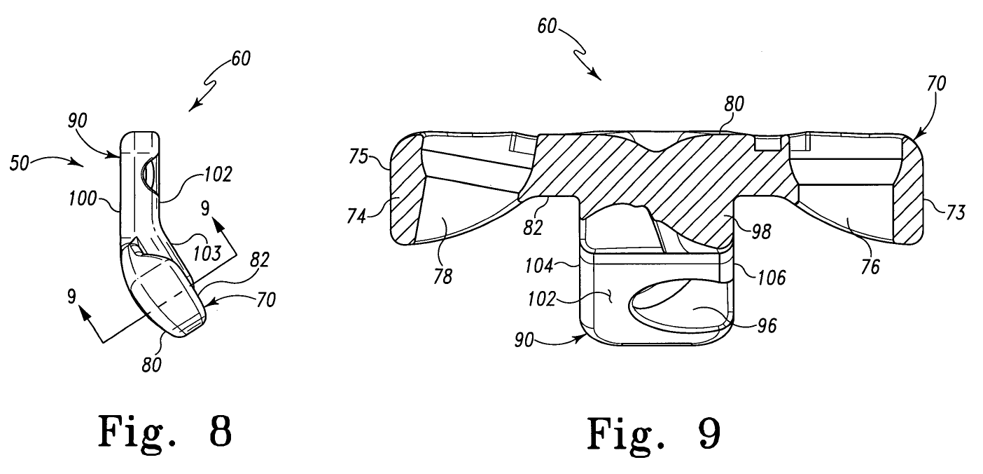

Referring to FIGS. 8-12, further details regarding plate member 60 will be discussed. As shown in FIGS. 8 and 9, bottom surface 102 of second portion 90 includes a central portion 103 at connecting portion 98 that overlaps or extends along a part of first portion 70, where it transitions to smoothly blend into bottom surface 82 of first portion 70. Accordingly, when first portion 70 is positioned extradiscally along vertebrae 32, 34 the bottom part of connecting portion 98 including central portion 103 of bottom surface 102 can extend into disc space 36.

Upper end portion 72 and lower end portion 74 each include a surface profile along bottom surface 82 that closely conforms to the outer surface profiles of vertebrae 32, 34 which, in the illustrated embodiment, is the antero-lateral aspect of vertebrae 32, 34. For example, first end portion 72 can be provided with a maximum thickness at upper end 73 and lower end portion 74 can be provided with a maximum thickness at lower end 75. Bottom surface 82 is convexly rounded along holes 76, 78 so that the thickness tapers toward connecting portion 98, where the thickness is then increased to accommodate the extension of central portion 103 of bottom surface 102 into the disc space.

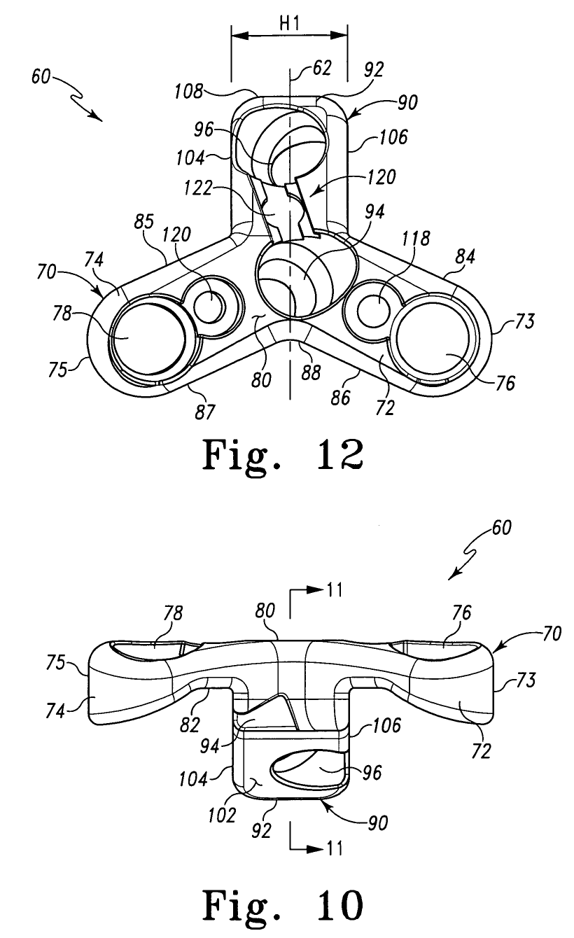

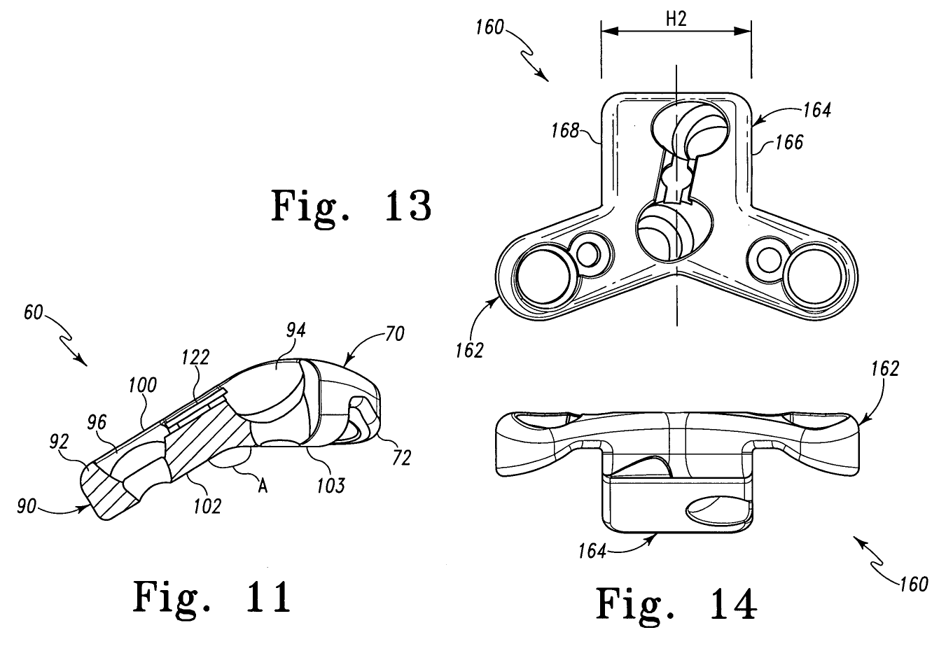

As shown in FIGS. 10-11, second portion 90 includes intradiscal portion 92 that is angled relative to first portion 70 so that intradiscal portion 92 can be located substantially completely within the disc space when first portion 70 is engaged to vertebrae 32, 34. For example, an angle A can be formed between central portion 103 of bottom surface 102 and the remaining portion of bottom surface 102 extending along intradiscal portion 92. In one embodiment angle A ranges from 120 degrees to 180 degrees. In another embodiment, angle A ranges from 135 degrees to 165 degrees. In still another embodiment, angle A is about 150 degrees. In still another embodiment, angle A is 148 degrees to provide an optimal antero-lateral fit with vertebrae 32, 34 and disc space 36 in a lumbar stabilization procedure of the L4 and L5 vertebrae.

Referring further to FIG. 12, second portion 90 includes an intradiscal side 108 located opposite first portion 70. Second portion 90 further includes an upper end 106 and a lower end 104 extending from first portion 70. Upper and lower ends 106, 104 can be separated by a height H1, and positionable adjacent an endplate of a respective one of the vertebrae 32, 34. In one embodiment, upper and lower ends 106, 104 contact the adjacent vertebral endplate to provide bearing support for the adjacent vertebrae in disc space 36.

First portion 70 includes upper end 73 and lower end 75. Intermediate sides 84, 85 extend from respective ones of the upper and lower ends 73, 75 and are angled toward intradiscal side 108 in the direction of medial-lateral axis 62 to the respective upper and lower ends 106, 104 of second portion 90. First portion 70 further includes upper and lower extradiscal sides 86, 87 extending from respective ones of upper and lower ends 73, 75. Extradiscal sides 86, 87 are angled toward second portion 90 in the direction of medial-lateral axis 62. A convexly curved transition surface 88 is provided between the upper and lower extradiscal sides 86, 87, and the angled extradiscal sides 86, 87 minimize protrusion of plate member 60 into the adjacent anatomy.

Second portion 90 extends from intermediate sides 84, 85 in a direction away from extradiscal sides 86, 87. Holes 94, 96 of second portion 90 can be aligned along medial-lateral axis 62. Medial-lateral axis 62 can bisect first portion 70 with holes 76, 78 lying on opposite sides of medial-lateral axis 62. It is further contemplated that either or both of first and second portions 70, 90 can extend a greater extent from one side of medial-lateral axis 62 than the other.

In the illustrated embodiment of FIGS. 1-12, plate member 60 includes a Y-shaped profile for screw placement and vertebral fixation provided by the relative orientations between the first and second portions 70, 90. First portion 70 forms an upper portion of the Y shape that is positioned so that the extradiscal sides 86, 87 are located extradiscally with transition surface 88 between vertebrae 32, 34. The lower leg of the Y shape opposite the upper portion is formed by second portion 90 and extends intradiscally so that intradiscal side 108 is located in the disc space.

Plate member 60 can further be provided with receptacles 118, 120 adjacent respective ones of the holes 76, 78 to receive respective ones of the retaining fasteners 112, 114. Receptacles 118, 120 are formed in plate member 60 adjacent to and opening at top surface 80. A retaining plate receptacle 122 can be provided adjacent top surface 100 of second portion 90 and extends between holes 94, 96 to receive retaining plate 116.

Referring now to FIGS. 13 and 14, there is shown another embodiment plate member 160 that is substantially identical to plate member 60. Plate member 160 includes a first portion 162 positionable extradiscally along vertebrae 32, 34 and a second portion 164 extending from a side of first portion 162 that is positioned intradiscally. Second portion 164 varies from second portion 90 of plate member 60 in that second portion 164 includes a height H2 between upper and lower ends 166, 168 thereof that is greater than the height H1 between upper and lower ends 106, 104 of second portion 90. Accordingly, the plate member with a height corresponding to the desired spacing between the endplates of vertebrae 32, 34 can be selected to provide a better fit in the disc space and also so that the second portion of the plate member can provide intervertebral support to maintain the disc space height. It is contemplated that various plate members can be provided in a kit with second portions having various heights from which the desired plate member can be selected prior to or during surgery.

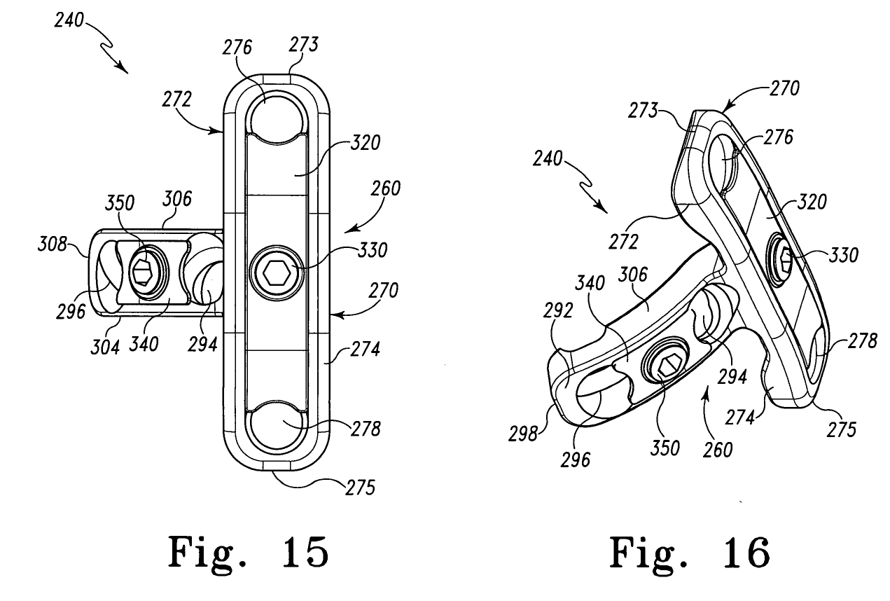

Referring now to FIGS. 15 and 16, there is shown another embodiment plating system 240 including a plate member 260. Plate member 260 includes a first portion 270 positionable extradiscally along adjacent vertebrae and a second portion 290 positionable intradiscally between adjacent vertebrae. As discussed above with respect to plate member 60, second portion 290 extends from an intermediate side of first portion 270 so that second portion 290 can receive anchors to intradiscally engage plate member 260 to the adjacent vertebrae with first portion 270 receiving anchors to extradiscally engage plate member 260 to the adjacent vertebrae.

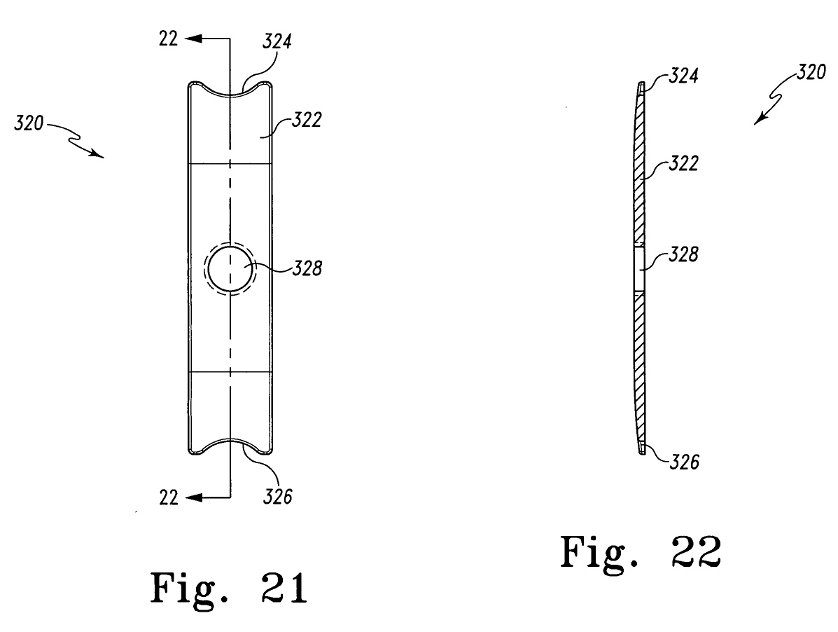

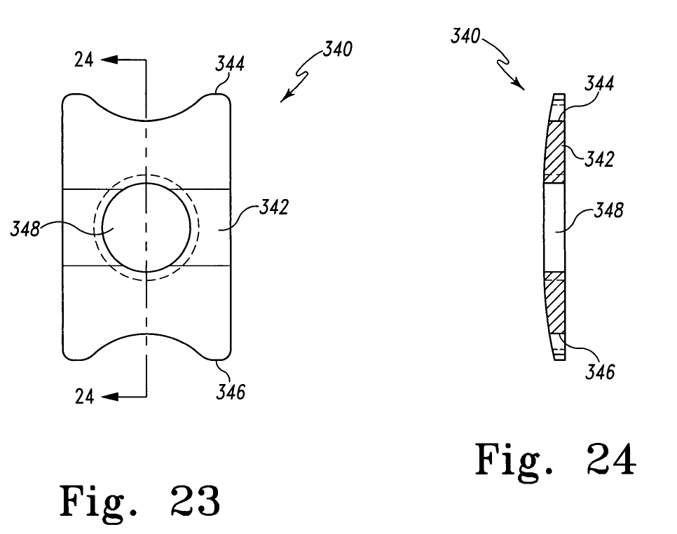

Plating system 240 further includes a first retaining plate 320 engaged to first portion 270 with a locking fastener 330, and a second retaining plate 340 engaged to second portion 290 with a locking fastener 350. As shown further in FIGS. 21-24, retaining plate 320 includes a body 322 extending between an upper end 324 and a lower end 326. A central bore 328 threadingly receives locking fastener 330, which engages a corresponding receptacle 316 in first portion 270 to secure retaining plate 320 thereto. When secured to plate member 260, upper end 324 is positioned adjacent to upper hole 276 in an at least partially overlapping arrangement, and lower end 326 is positioned adjacent to lower hole 278 in an least partially overlapping arrangement. The overlapping ends obstruct the adjacent hole to prevent an anchor positioned in the hole from backing out thereof. Each of the ends 322, 324 includes a concavely curved central portion to permit tool access thereby to engage the anchor should it be desired to do so when retaining plate 320 is engaged to plate member 260.

Retaining plate 340 is similar to retaining plate 320, but is positioned along second portion 290 in an at least partially overlapping arrangement with holes 294, 296. Retaining plate 340 includes a body 342 extending between opposite ends 344, 346. A central bore 348 threadingly receives locking fastener 350, which is engageable to receptacle 318 in second portion 290 to secure retaining plate 340 to second portion 290.

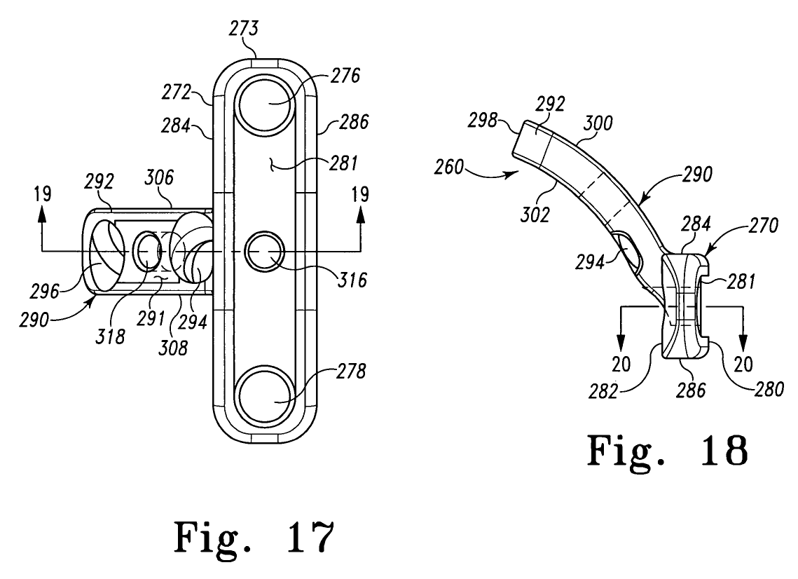

Referring to FIGS. 17-20, plate member 260 will be further discussed. Plate member 260 includes a T shape. First portion 270 forms an upper portion of the T shape, and extends extradiscally. Second portion 290 forms a lower leg of the T shape that is positioned at least partially intradiscally.

First portion 270 includes a top surface 280 and a bottom surface 282. A recess 281 is formed in top surface 280 to receive retaining plate 320 so that it does not substantially protrude from the top surface 280. Receptacle 316 is provided in recess 281 to receive locking fastener 330 to secure retaining plate 320 in recess 281. First portion 270 further includes an upper end portion 272 having an upper end 273 and an upper hole 276 extending between top and bottom surfaces 280, 282. An anchor 42 is positionable through upper hole 276 to secure first portion 270 to upper vertebra 32. First portion 270 further includes a lower end portion 274 having a lower end 275 and a lower hole 278 extending between top and bottom surfaces 280, 282. A second anchor 42 is positionable through lower hole 278 to secure first portion 270 to lower vertebra 34.

First portion 270 includes an extradiscal side 286 extending between top and bottom surfaces 280, 282 and upper and lower ends 273, 275. Opposite extradiscal side 286 is an intermediate side 284 extending between top and bottom surfaces 280, 282 and upper and lower ends 273, 275. Second portion 290 includes an intradiscal member 292 extending from first portion 270 from a location adjacent intermediate side 284 and in a direction away from extradiscal side 286. Intermediate side 284 extends along top surface 300 of second portion 290, providing a stepped configuration between first and second portions 270, 290 to offset second portion 290 into the disc space when first portion 270 is extradiscally engaged to the adjacent vertebrae.

Intradiscal member 292 includes a top surface 300 and a bottom surface 302. A recess 291 is formed in top surface 300 to receive retaining plate 320. Receptacle 318 receives locking fastener 350 that secures retaining plate 320 to second portion 290. Top and bottom surfaces 300, 302 can be curved to conform to the curvature around the perimeter of the adjacent vertebral endplates when positioned in the spinal disc space. A central hole 294 is provided through second portion 290 in a location adjacent first portion 270. Central hole 294 extends between and opens at top and bottom surfaces 300, 302. An intradiscal hole 296 is located adjacent an intradiscal side 298 of second portion 290. Holes 294, 296 can be angled cephaladly and caudally, respectively, or vice versa, so that at least one anchor engages respective ones of the upper and lower vertebrae 32, 34.

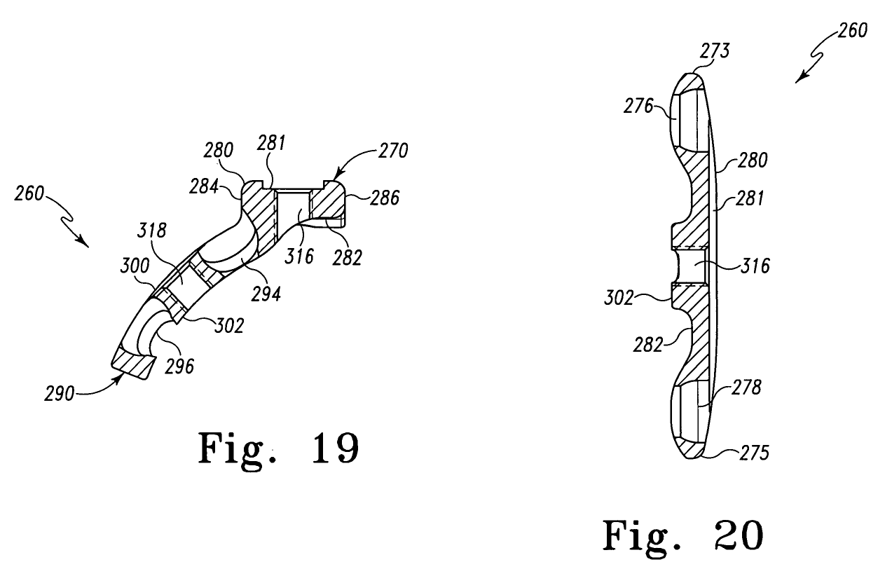

Intradiscal member 292 includes an upper end 306 positionable along an endplate of upper vertebra 32 and a lower end 308 positionable along an endplate of lower vertebra 34. Top surface 300 is offset below top surface 280, as shown in FIG. 19, to facilitate intradiscal placement of second portion 290. Bottom surface 302 extends at least partially along first portion 270 in the region of overlap between first and second portions 270, 290, and blends into bottom surface 282 without abrupt or sharp transitions.

Any of the plate member embodiments discussed herein can be made from any biocompatible material, including titanium, stainless steel, shape memory material, polymers, plastics, elastomers, ceramics, composites, bone, and combinations thereof. Placement of the plate members into the patient can be facilitated with a plate holder that releasably holds the plate member in a desired position during screw insertion. The plate holder may incorporate one or more guide members to guide hole formation and/or placement of the anchors through the plate holes. The plate holder can be configured to grab, hold, grip or engage one or more sides, ends or surfaces of one or both of the plate portions. The inserter can also be engageable to a hole or opening in the plate, such as receptacle 318 of plate member 260. In other embodiments, the plate member is top-loading on anchors engaged to the vertebrae. Other embodiments contemplate the plate member includes surface features, such as roughenings, spikes, ridges or other features that engage the adjacent vertebrae alone or in combination with the anchors.

The anchors can be bone screws with threaded shanks extending distally from an enlarged head. The shanks may be configured for self-drilling, self-tapping, or for insertion in drilled and tapped holes in the vertebrae. The head can include a spherical lower surface to facilitate pivoting movement relative to the plate member and recessing of the head relative to the top surface of the plate member. Other embodiments contemplate bone screws with other head configurations, including those that are not recessed relative to the top surface of the plate member. Still other embodiments contemplate anchors in forms other than bone screws, including suture anchors, bolts, spikes, cables, wires, bolts, hollow anchors for fusion, and cannulated anchors with or without fenestrations, for example.

While the invention has been illustrated and described in detail in the drawings and foregoing description, the same is to be considered as illustrative and not restrictive in character. All changes and modifications that come within the spirit of the invention are desired to be protected.

What is claimed is:

1. A spinal plating system, comprising:
a plate member having a first portion with a bottom surface positionable along first and second vertebrae and including first and second holes between a top surface and said bottom surface of said first portion, said first and second holes being configured to receive respective ones of first and second anchors outside a disc space between the first and second vertebrae to extradiscally engage said first portion to the first and second vertebrae, said first portion including an extradiscal side and an opposite intermediate side between said top and bottom surfaces, said plate member further including a second portion extending from said intermediate side of said first portion and away from said extradiscal side, wherein said second portion includes a top surface and a bottom surface and at least one hole extending between said top surface and bottom surface to receive a third anchor, said second portion being configured relative to said first portion for positioning in the disc space between the first and second vertebrae when said first portion is extradiscally engaged to the first and second vertebrae, said second portion being intradiscally engageable with at least one of the first and second vertebrae with said third anchor positioned through said third hole.

2. The system of claim 1, wherein said plate member includes a Y-shape.

3. The system of claim 1, wherein said plate member includes a T-shape.

4. The system of claim 1, further comprising at least one retaining mechanism to prevent back-out said anchors.

5. The system of claim 1, wherein said anchors are bone screws.

6. The system of claim 1, wherein said bottom surface of said first portion is contoured to conform to at least one of an antero-lateral and lateral profile of the first and second vertebrae.

7. The system of claim 6, wherein said second portion extends along a medial-lateral axis that bisects said first portion between said first and second holes.

8. The system of claim 7, wherein said bottom surface of said second portion forms an angle with said bottom surface of said first portion along said medial-lateral axis in the range from 120 degrees to 180 degrees.

9. The system of claim 1, further comprising fourth hole at a junction of said first and second portions, said fourth hole extending between said top and bottom surfaces of respective ones of said first and second portions.

10. The system of claim 9, wherein said third anchor engages one of the first and second vertebrae and a fourth anchor in said fourth hole engages the other of the first and second vertebrae.

11. The system of claim 9, wherein said second portion extends along a medial-lateral axis and said third and fourth holes are aligned along said medial-lateral axis.

12. The system of claim 11, wherein said medial-lateral axis bisects said first portion between said first and second holes.

13. The system of claim 1, wherein said second portion includes an upper end and a lower end, at least one of said upper and lower ends positionable adjacent an endplate of a respective one of the first and second vertebrae.

14. The system of claim 1, wherein said top surface of said second portion is recessed below said intermediate side of said first portion.

15. The system of claim 1, wherein said second portion extends from and interrupts said intermediate side.

16. A spinal plating system, comprising:
a plate member having a first portion and a second portion, each of said first and second portions including a top surface and a bottom surface extending between cephalad and caudal ends thereof, said first portion including an intermediate side and an opposite extradiscal side extending between said top and bottom surfaces thereof, said bottom surface of said first portion being positionable extradiscally along first and second vertebrae when said plate member is engaged to the first and second vertebrae, and said second portion extending from said intermediate side of said first portion and away from said extradiscal side with said cephalad and caudal ends of said second portion extending into a disc space between the first and second vertebrae when said first portion is extradiscally engaged to the first and second vertebrae.

17. The system of claim 16, wherein said first portion includes holes between said top surface and said bottom surface thereof, said holes receiving anchors to extradiscally engage said first portion to each of the first and second vertebrae.

18. The system of claim 17, wherein said second portion includes at least one hole extending between said top and bottom surfaces thereof to receive a third anchor, said second portion being intradiscally engageable with at least one of the first and second vertebrae with said third anchor positioned through said third hole.

19. The system of claim 18, wherein said second portion includes a fourth hole extending between said top and bottom surfaces thereof, said fourth hole receiving a fourth anchor to intradiscally engage said second portion to the other of the first and second vertebrae.

20. The system of claim 16, wherein said second portion includes at least one hole extending between said top and bottom surfaces thereof to receive an anchor, said second portion being intradiscally engageable with at least one of the first and second vertebrae with said anchor positioned through said at least one hole.

21. The system of claim 20, wherein said first portion includes holes between said top surface and said bottom surface thereof, said holes receiving anchors outside the disc space to extradiscally engage said first portion to each of the first and second vertebrae.

22. The system of claim 16, wherein said plate member includes a Y-shape.

23. The system of claim 16, wherein said plate member includes a T-shape.

24. The system of claim 16, said bottom surface of said first portion is contoured to conform to an antero-lateral profile of the first and second vertebrae.

25. The system of claim 16, wherein said second portion extends along a medial-lateral axis that bisects said first portion between said cephalad and caudal ends.

26. The system of claim 25, wherein said top and bottom surfaces of said second portion are curved along said medial-lateral axis.

27. The system of claim 16, wherein at least one of said cephalad and caudal ends of said second portion are positionable in contact with an endplate of a respective one of the first and second vertebrae.

28. A spinal plating system, comprising:
a plate member having a first portion positionable extradiscally along first and second vertebrae and engageable thereto at a location offset laterally from a sagittal plane of the first and second vertebrae and a second portion extending in a medial-lateral direction from said first portion and positionable intradiscally between the first and second vertebrae, said second portion including at least one hole extending therethrough for receiving an anchor to intradiscally engage said second portion to at least one of the first and second vertebrae at a location adjacent the sagittal plane.

29. The system of claim 28, wherein said second portion includes a second hole extending therethrough adjacent said first portion, said second hole receiving a second anchor for engaging said second portion to the other of the first and second vertebrae.

30. The system of claim 29, wherein said second portion extends along a medial-lateral axis and said holes of said second portion are aligned along said medial-lateral axis.

31. The system of claim 30, wherein said first portion includes first and second holes for receiving anchors to extradiscally engage respective ones of the first and second vertebrae, said first and second holes of said first portion being offset on opposite sides of said medial-lateral axis.

32. The system of claim 28, wherein said first portion includes first and second holes therethrough for receiving anchors to engage said first portion to respective ones of the first and second vertebrae.

33. The system of claim 28, wherein said first portion includes a bottom surface positionable along the first and second vertebrae and an opposite top surface, an upper end positionable along the first vertebrae and an opposite lower end positionable along the lower vertebra, and an extradiscal side and an opposite intermediate side extending between said top and bottom surfaces and said upper and lower ends.

34. The system of claim 33, wherein said second portion extends from said intermediate side in a direction away from said extradiscal first side.

35. The system of claim 34, wherein said second portion includes an upper end positionable along an endplate of the first vertebra and a lower end positionable along the endplate of the second vertebra.

36. A spinal plating system, comprising:
a plate member having a Y-shape, wherein a first portion of said plate member corresponds to an upper portion of said Y-shape and is positionable extradiscally between adjacent vertebra and engageable to the adjacent vertebrae with respective ones of first and second anchors and a second portion of said plate member corresponds to a lower leg of said Y-shape and is positionable intradiscally between the adjacent vertebrae and engageable to at least one of the adjacent vertebrae with a third anchor.

37. The system of claim 36, wherein said second portion includes first and second holes therethrough to receive respective ones of said third anchor and a fourth anchor to engage said second portion to respective ones of the adjacent vertebrae from within the disc space between the adjacent vertebrae.

38. The system of claim 37, further comprising:
first and second retaining mechanisms engaged to said plate member adjacent respective ones of first and second anchors of said first portion; and
a third retaining mechanism engaged to said plate member and extending between said third and fourth anchors of said second portion.

39. A method for stabilizing first and second vertebrae of a spinal column, comprising:
providing a plate member having a first portion and a second portion extending from a side of the first portion;
positioning the first portion extradiscally along the first and second vertebrae at a location offset laterally from a sagittal plane of the first and second vertebrae with the second portion in the disc space between the first and second vertebrae;
engaging the first portion to each of the first and second vertebrae with respective ones of first and second anchors; and
engaging the second portion to at least one of the first and second vertebrae with a third anchor extending through an endplate of the at least one of the first and second vertebrae.

40. The method of claim 39, further comprising engaging the second portion to the other of the first and second vertebrae with a fourth anchor through an endplate of the other of the first and second vertebrae.

41. A method for stabilizing first and second vertebrae of the spinal column, comprising:
positioning a bottom surface of a first portion of a plate member extradiscally along first and second vertebrae;
positioning a second portion of the plate member intradiscally between the first and second vertebrae, wherein the second portion extends from a side of first portion extending adjacent said bottom surface;
extradiscally engaging anchors through the first portion to each of the first and second vertebrae; and
intradiscally engaging at least one anchor through the second portion to at least one of the first and second vertebrae.

42. The method of claim 41, wherein engaging the second portion includes intradiscally engaging two anchors through the second portion to respective ones of the first and second vertebrae.

43. The method of claim 41, wherein positioning the first portion includes positioning the first portion laterally along the first and second vertebrae with the second portion extending in an antero portion of a disc space between the first and second vertebrae and toward a sagittal plane of the first and second vertebrae.

44. The method of claim 43, wherein engaging the second portion includes positioning anchors through the second portion and endplates of the first and second vertebrae in a direction generally parallel to the sagittal plane.

45. The method of claim 44, wherein engaging the first portion includes positioning anchors through the first portion and into the first and second vertebrae in a direction that intersects the sagittal plane.

46. The method of claim 41, wherein positioning the first portion includes positioning the first portion antero-laterally along the first and second vertebrae with the second portion extending along an anterior portion of a disc space between the first and second vertebrae.
